(19) United States
(12) Patent Application Publication
(10) Pub. No.: US 2003/0022302 A1
(43) Pub. Date: Jan. 30, 2003

(54) Toll-like receptor

(76) Inventors: Alan Peter Lewis, Stevenage (GB); Keith Paul Ray, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/181,800

(22) PCT Filed: Jan. 25, 2001
(86) PCT No.: PCT/GB01/00299

(30) Foreign Application Priority Data
Jan. 25, 2000 (GB) 0001704.6

(51) Int. Cl. C12P 21/02; C12N 5/06; C07H 21/04; C07K 14/705

(52) U.S. Cl. 435/69.1; 435/320.1; 435/325; 530/350; 536/23.2

(57) ABSTRACT

An isolated Toll-like-receptor polypeptide comprises the amino acid sequence of SEQ ID NO: 2, a variant or a fragment thereof which has immunomodulatory activity. Polynucleotides encoding such a Toll-like receptor are also described. A method for identification of a substance that modulates Toll-like receptor activity comprises contacting a polypeptide of the invention with a test substance and monitoring for immunomodulatory activity.

### FIELD OF THE INVENTION

The present invention relates to a novel Toll-like receptor or a variant thereof. A variant may demonstrate Toll-like receptor activity such as activation of NFκB, or may inhibit Toll-like receptor activity.

### BACKGROUND OF THE INVENTION

A family of human Toll-like receptors has been described in the literature. These receptors are termed Toll-like receptors in view of common structural features shared with a Drosophila Toll (dToll) receptor molecule which is involved in embryonic development. Toll and Toll-Like receptors are type I transmembrane proteins, with extracellular leucine rich repeat motifs and an intracellular signalling domain homologous to that of members of the interleukin 1 receptor superfamily.

Drosophila Toll also plays an important role in the adult fly and is involved in immune surveillance mechanisms required for recognition of bacterial and fungal pathogens and regulation of specific innate immune defense gene expression. Activation of dToll receptors in response to infection by specific micro-organisms is thought to require the production of a protein ligand called Spaetzle. The human Toll-like receptors (hTLRs) are also thought to participate in mechanisms of innate immunity and inflammation acting as pattern recognition receptors (PRRs) for bacteria and other micro-organisms. hTLRs are expressed on antigen presenting cells including monocytes and dendritic cells and like dToll play roles in innate immunity. TLRs can elicit pro-inflammatory cytokine production and induce expression of cell surface co-stimulatory receptors required for activation of T-cells. Some hTLRs may help to co-ordinate interactions between cells of the innate and acquired immune systems to orchestrate an integrated immune response to infection.

The specific functions of two mammalian TLRs, TLR2 and TLR4, have recently been identified. TLR2 and TLR4 are involved in mediating host responses to gram positive and gram negative bacteria through recognition of specific bacterial wall components. It has also recently been shown that TLR4 mediates responses to certain viral proteins including respiratory syncytial virus (RSV) (Nature Immunology 1: 398 2000).

Additionally TLRs may form heterodimeric functional complexes. Components of the intracellular signal transduction pathways of some hTLRs appear to be shared with interleukin-1 (IL-1) receptor transduction pathways. Stimulation of TLR2 and TLR4 leads to activation of NFκB via an adapter protein MyD88 and recruitment of the IL-1 receptor associated kinases (IRAKs).

### SUMMARY OF THE INVENTION

A novel Toll-like receptor is now provided which is a screening target for the identification and development of novel pharmaceutical agents which modulate the activity of the receptor and in particular have immunomodulatory activity. These agents may be used in the treatment and/or prophylaxis of inflammatory diseases, cardiovascular diseases, systemic infections and autoimmune diseases, such as asthma, rhinitis, chronic obstructive pulmonary disease (COPD), emphysema, inflammatory bowel disease such as ulcerative colitis and Crohn's disease, rheumatoid arthritis, osteoarthritis, psoriasis, Alzheimers disease, atherosclerosis, viral, fungal and bacterial infections, septic shock syndrome associated with systemic infection involving gram positive and gram negative bacteria, diabetes, Multiple Sclerosis. These agents may also be used as immunoadjuvants to enhance or alter the immune response in vaccine therapy.

Accordingly, the present invention provides an isolated Toll-like-receptor polypeptide which comprises:

(i) the amino acid sequence of SEQ ID NO: 2;

(ii) a variant of (i) which has immunomodulatory activity; or

(iii) a fragment of (i) or (ii) which retains immunomodulatory activity.

Preferably, a variant has at least 80% identity to the amino acid sequence of SEQ ID NO: 2, more preferably at least 95% identity therewith, for example 97% identity therewith.

The invention also provides a polynucleotide encoding a polypeptide of the invention. Such a polynucleotide may be a polynucleotide which encodes a Toll-like receptor polypeptide which has immunomodulatory activity. The polynucleotides of the invention may be DNA or RNA, for example mRNA. A polynucleotide according to the invention comprises:

(a) the nucleic acid sequence of SEQ ID NO: 1 and/or a sequence complementary thereto;

(b) a sequence which hybridises under stringent conditions to a sequence as defined in (a);

(c) a sequence that is degenerate as a result of the genetic code to a sequence as defined in (a) or (b); or

(d) a sequence having at least 60% percent identity to a sequence as defined in (a), (b) or (c).

The present invention also provides a polypeptide expressed from a polynucleotide according to (a), (b), (c) or (d) above, in particular a polypeptide comprising a toll-like receptor according to the invention, encoded by the mRNA derived from a DNA sequence according to (a) or (b) above, thus the invention provides an isolated toll-like receptor polypeptide which is obtainable by expression in vitro or in vivo of a DNA molecule comprising the sequence of nucleotides as shown in SEQ ID NO. 1.

The polypeptides of SEQ ID NO 2 and SEQ ID NO 4, which are different isoforms expressed from the multiple exon tlr9 gene, are herein referred to as TLR9 and TLR9-A, respectively. TLR9-A is encoded by the nucleotide sequence of SEQ ID NO. 3, which is encoded within SEQ ID NO. 1 except for the initiating methionine, that is encoded by a second exon as illustrated in FIG. 1. (see Hemmi et. al. Nature 408, 740-745 2000; Du et al, European Cytokine Network, 11: 362-371, 2000; Chuang and Ulevitch, European Cytokine Network, 11: 372-378 for isolation of the cDNAs, and corresponding sequence database accessions EMBL:AB045180, EMBL:AF259262, EMBL:AF245704).

In further aspects of the invention we provide:

an expression vector capable of expressing a polypeptide of the invention comprising a polynucleotide as defined above.

a host cell comprising an expression vector of the invention.

an antibody specific for a polypeptide of the invention.

a method for identification of a compound that modulates Toll-like receptor activity, comprising contacting a polypeptide of the invention with a test compound and monitoring for immunomodulatory activity.

Compounds which are identifiable in accordance with this method may be used in the treatment of a subject having a disorder that is responsive to Toll-like receptor modulation such as an inflammatory or cardiovascular disorder or systemic infection or autoimmune disease, including asthma, chronic obstructive pulmonary disease (COPD), emphysema, inflammatory bowel disease such as ulcerative colitis and Crohn's disease, rheumatoid arthritis, osteoarthritis, psoriasis, viral, fungal and bacterial infections, Alzheimers disease, atherosclerosis, septic shock syndrome associated with systemic infection involving gram positive and gram negative bacteria, diabetes and Multiple Sclerosis. In particular, compounds which are identifiable in accordance with this method may be used in the treatment of a subject having allergic asthma or rhinitis. Further, such compounds may have immunomodulatory activity and be of use in the treatment of, or as adjuvants in vaccination against, bacterial or viral infections or as components of anti-cancer vaccines.

Compounds identifiable in accordance with this method include, in particular, synthetic or naturally occurring oligopeptides or polypeptides, oligonucleotides or polynucleotides which bind directly to the Toll-like receptor of the invention, and synthetic or naturally occurring oligopeptides or polypeptides, oligonucleotides or polynucleotides which modulate the Toll-like receptor of the present invention via one or more intermediate signal transducers. Such oligo- or polynucleotides may be “CG-rich” sequences or sequences including one or more unmethylated CpG nucleotide pairs.

In an alternative aspect of the invention, a polypeptide comprises a fragment or variant of SEQ ID NO 2 which is capable of inhibiting the activity of TLR9 or TLR9-A, for use in the treatment of an immune or inflammatory disorder.

In a further aspect of the invention, a polypeptide or polynucleotide in accordance with the invention or a compound identifiable in accordance with the invention is provided for use as an adjuvant or as an immunotherapeutic agent, for example in a vaccine.

### Brief Description of the Sequences

SEQ ID NO: 1 is the amino acid sequence of human protein TLR9 and its encoding DNA;

SEQ ID NO; 2 is the amino acid sequence alone of TLR9;

SEQ ID NO: 3 is the amino acid sequence of human protein TLR9-A and its encoding cDNA (EMBL:AF259262);

SEQ ID NO: 4 is the amino acid sequence alone of TLR9-A (Hemmi et al.).

### BRIEF DESCRIPTION OF THE DRAWINGS

FIG. 1 is a diagrammatic illustration showing the exon arrangement encoding TLR9 and TLR9-A;

FIG. 2 shows tissue distribution data for TLR9 (using a human tissue cDNA plate). The profile shows predominant expression in tonsil and adenoid tissues with lower levels of expression detected in adipose, adrenal, foetal brain, cerebellum, jejunum, lung, myometrium, omentum, head of pancreas, rectum, skeletal muscle, spleen and thymus tissues;

FIG. 3 shows tissue distribution data for TLR9 (using a human disease cDNA plate). The profile shows predominant expression in lung tissue, bone marrow and PBMC with lower levels of expression detected in some colon, breast and brain/cerebellum samples;

FIG. 4 illustrates, in diagrammatic form, the predicted structural topology of human TLR9—“TM” is the transmembrane portion, “TIR” is the cytosolic region conserved among interleukin and toll-like receptors known as the Toll Interleukin Receptor domain.

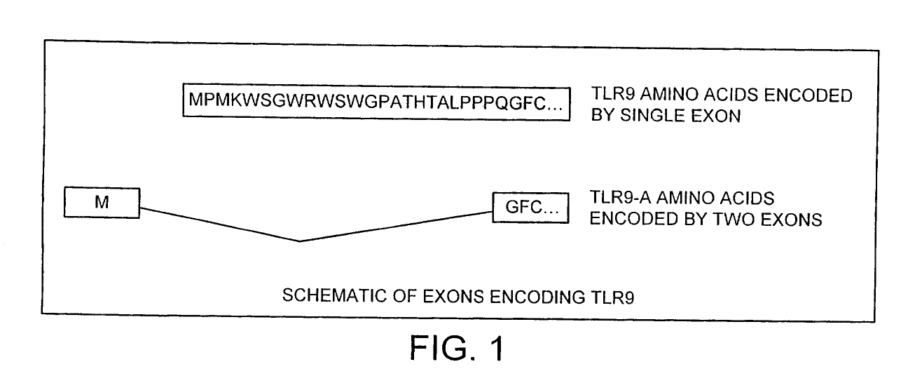

### DETAILED DESCRIPTION OF THE INVENTION

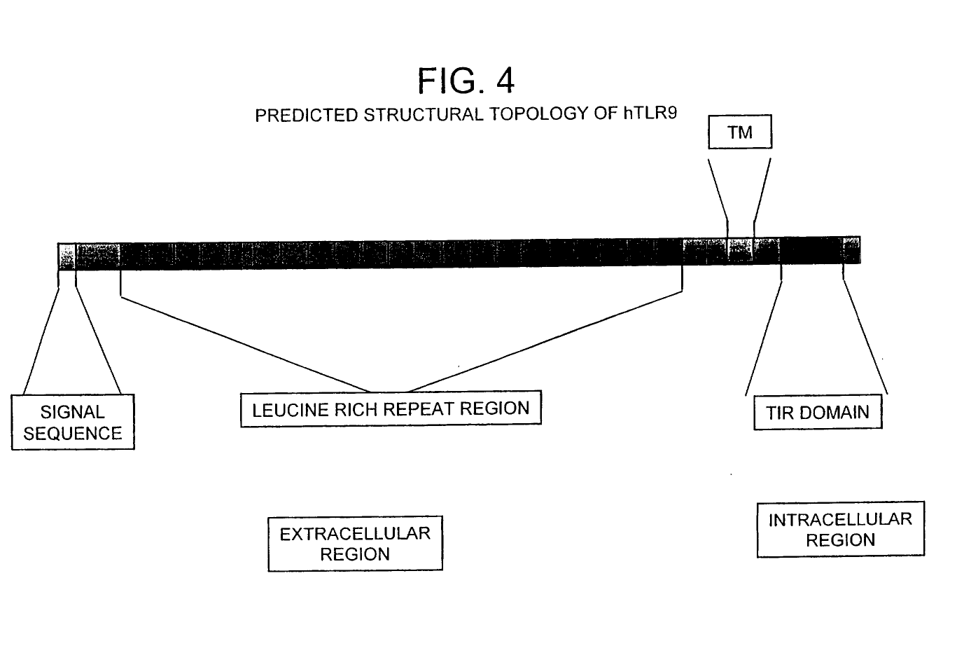

A single open reading frame was identified in genomic DNA (SEQ ID NO. 1), which encodes a protein of 1055 residues, predicted molecular weight 118,515 (PeptideSort—GCG Software) and the amino acid sequence shown in SEQ ID NO. 2. This sequence included the TIR domain common to Toll-like receptors and members of the interleukin-1 receptor family e.g. IL1RI, and the N-terminal sequence contains structural features as shown in FIG. 4. These features include, in order from the N- to the C-terminus, a predicted signal sequence with a potential cleavage site between residues 48 and 49 or 50 and 51 (SPScan in GCG; SignalP), a leucine-rich repeat motif domain, a potential transmembrane region and the Toll/IL-1R homologous region (TIR; Pfam Database). These motifs confirmed that the protein was likely to be expressed as a single transmembrane receptor-like molecule belonging to the TLR rather than the IL1R family and therefore, it was designated TLR9 based on existing published and in-house nomenclature.

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The present invention relates to a human Toll-like receptor, referred to herein as TLR9, and variants or fragments thereof. Sequence information for TLR9 is provided in SEQ ID NO: 1 (nucleotide and amino acid) and in SEQ ID NO: 2 (amino acid only). A polypeptide of the invention consists essentially of the amino acid sequence of SEQ ID NO: 2 or of a functional variant of that sequence. One important variant of TLR9 is TLR9-A, sequence information for which is provided in SEQ ID NO: 3 (nucleotide and amino acid) and in SEQ ID NO: 4 (amino acid only).

The polypeptides are provided in isolated form. The term “isolated” is intended to convey that the polypeptide is not in its native state, insofar as it has been purified at least to some extent or has been synthetically produced, for example by recombinant methods. The polypeptide may be mixed with carriers or diluents which will not interfere with the intended purpose of the polypeptide and still be regarded as substantially isolated. The term “isolated” therefore includes the possibility of the polypeptide being in combination with other biological or non-biological material, such as cells, suspensions of cells or cell fragments, proteins, peptides, expression vectors, organic or inorganic solvents, or other materials where appropriate, but excludes the situation where the polypeptide is in a state as found in nature.

A polypeptide of the invention may also be in a substantially purified form, in which case it will generally comprise the polypeptide in a preparation in which more than 50%, e.g. more than 80%, 90%, 95% or 99%, by weight of the polypeptide in the preparation is a polypeptide of the invention. Routine methods, can be employed to purify and/or synthesise the proteins according to the invention. Such methods are well understood by persons skilled in the art, and include techniques such as those disclosed in Sambrook et al, Molecular Cloning: a Laboratory Manual, 2^{nd} Edition, CSH Laboratory Press (1989), the disclosure of which is included herein in its entirety by way of reference.

The term “variants” refers to polypeptides which have the same essential character or basic biological functionality as TLR9. The essential character of TLR9 can be defined as that of a Toll-like receptor. In particular, it refers to a polypeptide which has an immunomodulatory activity. In one aspect of the invention, a polypeptide of the invention may activate NFκB or may elicit pro-inflammatory cytokine production or induce expression of cell surface co-stimulatory receptors required for activation of T-cells.

Alternatively, a variant of the polypeptide of the invention is one which exhibits binding to the same ligand as TLR9. Such ligand binding may be assayed using the assays described below.

In other aspects of the invention a variant is one which does not show the same function as TLR9 but which may be used to inhibit this function. For example, a variant polypeptide for use in an assay or therapy is one which inhibits TLR9 activity, for example by inhibiting or competing out ligand binding or receptor complex formation by TLR9. Alternatively, a variant may be one which inhibits ligand binding to TLR9. Such a variant may inhibit activation of NFκB or inhibit cytokine production and expression of cell surface co-stimulatory receptors. Such inhibitors may be used as immunomodulators to reduce inappropriate TLR activation in asthma or other chronic inflammatory diseases, or septic shock.

To determine whether a variant has the same essential function as TLR9, the immunomodulatory activity can be determined by monitoring the effect of a substance on different immune responses. For example the effect of the substance under test on NFκB activation mediated through binding the polypeptide of the present invention may be monitored. This can be carried out, for example, by co-transfection of a construct expressing the polypeptide with a construct containing a reporter gene, such as secreted placental alkaline phosphatase, under the control of a suitable NFκB-responsive promoter and monitoring for expression of the reporter gene.

Alternatively, other immunomodulatory activity such as the production of cytokines can be determined by monitoring cytokine production following incubation of a test substance with a cell expressing a polypeptide of the invention. Such assays may be carried out in the presence or absence of additional T-lymphocytes to assess the effect of such cytokines, or the direct action of a polypeptide of the invention, on such T-lymphocytes to thus determine immunomodulatory activity.

Alternatively, the Toll-like receptor functionality is as a peptide which binds a ligand of TLR9, inhibits immunomodulatory activity by TLR9 or inhibits ligand binding to TLR9 and can be determined by an assay as described below.

Preferably, a polypeptide of the invention will show the structural features associated with a Toll-like receptor. Preferably, a polypeptide of the invention, or a functional fragment thereof, contains one or more of the following structural features associated with a Toll-like receptor: an extracellular region containing leucine-rich repeat motif and cysteine-rich regions involved in ligand binding; a single hydrophobic transmembrane region; and a C-terminal cytoplasmic domain sharing homology with other TLRs and members of the IL-1 receptor family.

Typically, polypeptides with more than about 65% identity, preferably at least 80% or at least 90% and particularly preferably at least 95%, at least 97%, or at least 99% identity, with the amino acid sequences of SEQ ID NO: 2 over a region of at least 20, preferably at least 30, at least 40, at least 60 or at least 100 contiguous amino acids or over the full length of SEQ ID NO: 2, are considered as variants of the proteins. Identity is calculated using the widely used GCG (University of Wisconsin) suite of programs and preferably using the distances software (correction method). Such variants may include allelic variants and the deletion, modification or addition of single amino acids or groups of amino acids within the protein sequence, as long as the peptide maintains the basic biological functionally of the Toll-like receptor, having a similar function to TLR9 or inhibits such function such as preventing ligand binding or TLR9 mediated activation. Such variants also include isoforms such as TLR9-A, which is 23 amino acids (or 2.2%) shorter than TLR9 (see SEQ ID NO: 2 and SEQ ID NO: 4) and thus shows 97.8 identity therewith. Transcription of the nucleotide sequence presented in SEQ ID NO: 1 can result, due to variable mRNA splicing involving a second exon encoding an alternative initiating methionine, in an mRNA having the sequence of SEQ ID NO: 3 which, when translated, results in the polypeptide TLR9-A depicted in SEQ ID NO: 4.

Amino acid substitutions may be made, for example from 1, 2 or 3 to 10, 20 or 30 substitutions. The modified polypeptide generally retains activity as a TLR9 receptor or inhibitor of TLR9 receptor activity. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Shorter polypeptide sequences are within the scope of the invention. For example, a peptide of at least 20 amino acids or up to 50, 60, 70, 80, 100 or 150 amino acids in length is considered to fall within the scope of the invention as long as it demonstrates the basic biological functionality of TLR9 or inhibits TLR9. In accordance with this aspect of the invention the peptide may also comprise an epitope of TLR9 for generation of antibodies. In particular, but not exclusively, this aspect of the invention encompasses the situation when the protein is a fragment of the complete protein sequence and may represent a ligand-binding region (N-terminal extracellular domain) or an effector binding region (C-terminal intracellular domain). Fragments from which the C-terminus has been removed may be used as decoy receptors. Other fragments such as a secreted or soluble form of the receptor may be generated for use in an assay or in therapy in accordance with the invention. Such fragments can also be used to raise anti-TLR9 antibodies.

Polypeptides of the invention may be chemically modified, e.g. post-translationally modified. For example, they may be glycosylated or may comprise modified amino acid residues. They may also be modified by the addition of histidine residues or an epitope tag for example by a (His) 8 or (His) 6 sequence or a HA, T7, Myc or Flag tag to assist their purification or detection. They may be modified by the addition of a signal sequence to promote insertion into the cell membrane. Such modified polypeptides fall within the scope of the term “polypeptide” of the invention.

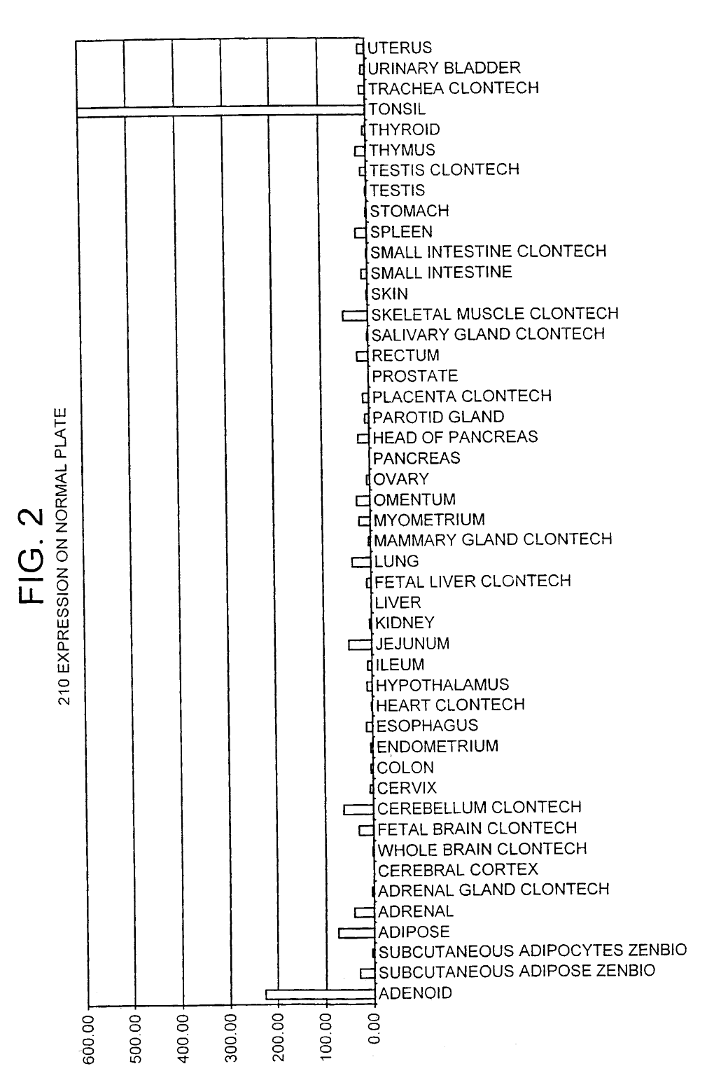

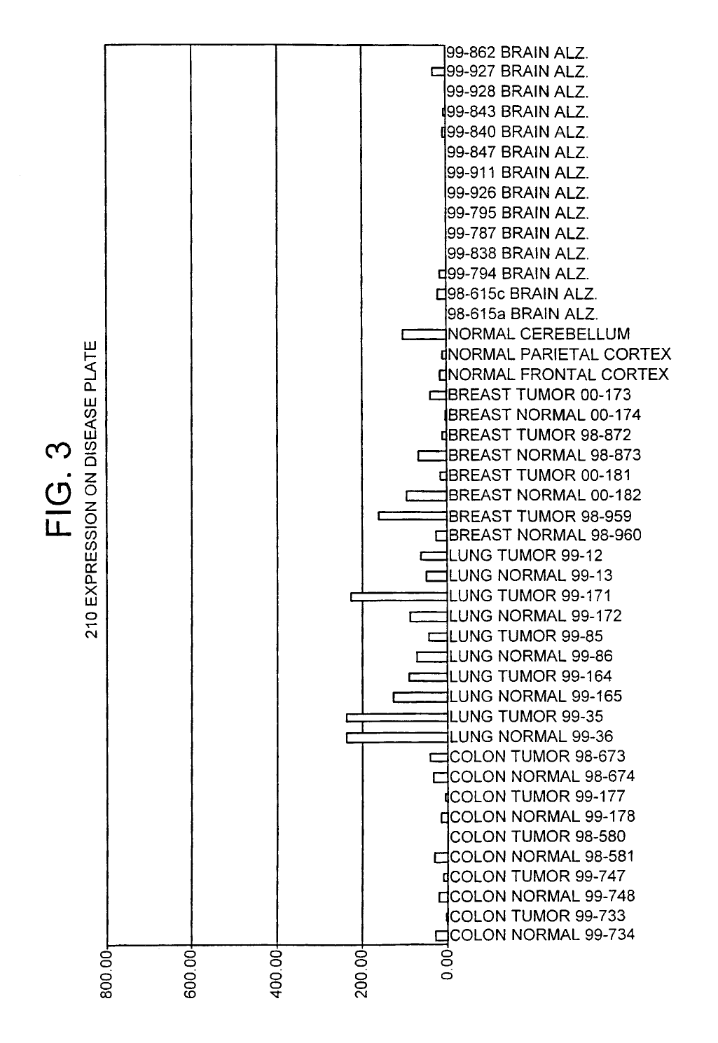

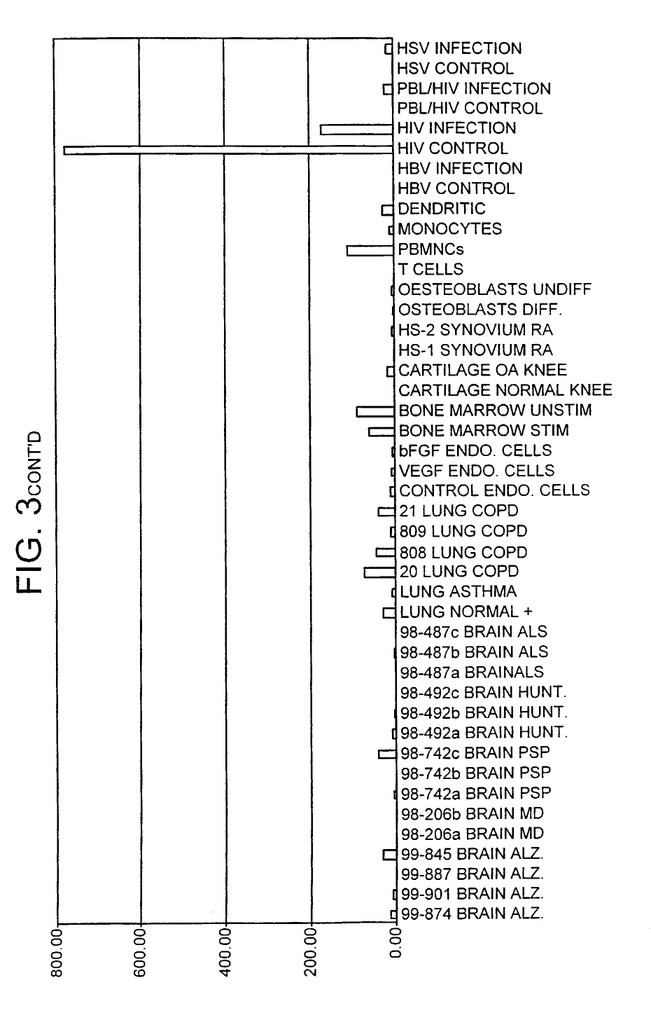

The invention also includes nucleotide sequences that encode for TLR9 or variants thereof as well as nucleotide sequences which are complementary thereto. The nucleotide sequence may be RNA or DNA including genomic DNA, synthetic DNA or cDNA. Preferably the nucleotide sequence is a DNA sequence and most preferably, a cDNA sequence. Nucleotide sequence information is provided in SEQ ID NO: 1. Such nucleotides can be isolated from human cells or synthesised according to methods well known in the art, as described by way of example in Sambrook et al. Such nucleotides can typically be isolated from activated cells of the immune system, heart, lung, pancreatic islet cells and lymph nodes, adenoid and tonsil tissues. FIGS. 2 and 3 show the tissue distribution of RNA encoding TLR9, as determined by extraction of total RNA from normal or disease tissue or cells which is then used to generate cDNA for real time quantitative PCR using suitable primers and probes (TaqMan analysis) to assess expression patterns. The profiles show differential expression across tissues tested and predominance to sites containing inflammatory cell types.

Typically a polynucleotide of the invention comprises a contiguous sequence of nucleotides which is capable of hybridising under selective conditions to the coding sequence or the complement of the coding sequence of SEQ ID NO: 1.

A polynucleotide of the invention can hybridize to the coding sequence or the complement of the coding sequence of SEQ ID NO: 1 (or of SEQ ID NO: 3) at a level significantly above background. Background hybridisation may occur, for example, because of other cDNAs present in a cDNA library. The signal level generated by the interaction between a polynucleotide of the invention and the coding sequence or complement of the coding sequence of SEQ ID NO: 1 or of SEQ ID NO: 3 is typically at least 10 fold, preferably at least 100 fold, as intense as interactions between other polynucleotides and the coding sequence of SEQ ID NO: 1 or of SEQ ID NO: 3. The intensity of interaction may be measured, for example, by radiolabelling the probe, e.g. with ³²P, Selective hybridisation may typically be achieved using conditions of low stringency (0.03M sodium chloride and 0.03M sodium citrate at about 40° C.), medium stringency (for example, 0.03M sodium chloride and 0.03M sodium citrate at about 50° C.) or high stringency (for example, 0.03M sodium chloride and 0.03M sodium citrate at about 60° C.).

The coding sequence of SEQ ID NO: 1 may be modified by nucleotide substitutions, for example from 1, 2 or 3 to 10, 25, 50 or 100 substitutions. The polynucleotides of the present invention may alternatively or additionally be modified by one or more insertions and/or deletions and/or by an extension at either or both ends. The modified polynucleotide generally encodes a polypeptide which has Toll-like receptor activity or inhibits the activity of TLR9. Degenerate substitutions may be made and/or substitutions may be made which would result in a conservative amino acid substitution when the modified sequence is translated, for example as shown in the Table above.

A nucleotide sequence of the invention which is capable of selectively hybridising to the complement of the DNA coding sequence of SEQ ID NO: 1 will generally have at least 60%, at least 70%, at least 80%, at least 90%, at least 95%, at least 98% or at least 99% sequence identity to the coding sequence of SEQ ID NO: 1 over a region of at least 20, preferably at least 30, for instance at least 40, at least 60, more preferably at least 100 contiguous nucleotides or most preferably over the full length of SEQ ID NO: 1. Methods of measuring nucleic acid and protein homology are well known in the art. For example the UWGCG Package provides the BESTFIT program which can be used to calculate homology (Devereux et al 1984). Similarly the PILEUP and BLAST algorithms can be used to line up sequences (for example are described in Altschul 1993, and Altschul et al 1990). Many different settings are possible for such programs. In accordance with the invention, the default settings may be used.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides. The most preferred sequences have at least 70% sequence identity over at least 70% of the full length of the sequence provided by SEQ ID NO: 1.

The nucleotides according to the invention have utility in production of the proteins according to the invention, which may take place in vitro, in vivo or ex vivo. The nucleotides may be involved in recombinant protein synthesis or indeed as therapeutic agents in their own right, utilised in gene therapy techniques. Nucleotides complementary to those encoding TLR9, or antisense sequences, may also be used in gene therapy, such as in strategies for down regulation of expression of the proteins of the invention.

Polynucleotides of the invention may be used as a primer, e.g. a PCR primer, a primer for an alternative amplification reaction, a probe e.g. labelled with a revealing label by conventional means using radioactive or non-radioactive labels, or the polynucleotides may be cloned into vectors.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500 nucleotides in length, or even up to a few nucleotides, such as five or ten nucleotides, short of the coding sequence of SEQ ID NO: 1.

The present invention also includes expression vectors that comprise nucleotide sequences encoding the proteins or variants thereof of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to a person skilled in the art. By way of further example in this regard we refer to Sambrook et al.

Polynucleotides according to the invention may also be inserted into the vectors described above in an antisense orientation in order to provide for the production of antisense RNA. Antisense RNA or other antisense polynucleotides may also be produced by synthetic means. Such antisense polynucleotides may be used as test compounds in the assays of the invention or may be useful in a method of treatment of the human or animal body by therapy.

Preferably, a polynucleotide of the invention or for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example in a method of gene therapy.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

Mammalian promoters, such as β-actin promoters, may be used. Tissue-specific promoters may be used. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide which comprise sequences homologous to eukaryotic genomic sequences, preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses (such as HPV-16 or HPV-18). Gene transfer techniques using these viruses are known to those skilled in the art. Retrovirus vectors for example may be used to stably integrate the polynucleotide giving rise to the RNA into the host genome. Replication-defective adenovirus vectors by contrast remain episomal and therefore allow transient expression.

The invention also includes cells that have been modified to express the TLR9 polypeptide or a variant thereof. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors encoding for a polypeptide according to the invention include mammalian HEK293T, CHO, HeLa and COS cells. Preferably the cell line selected will be one which is not only stable, but also allows for mature glycosylation and cell surface expression of a polypeptide. Cells such as T-cells, monocytes or dendritic cells expressing the receptor may be used for example in screening. Expression may be achieved in transformed oocytes. A polypeptide of the invention may be expressed in cells of a transgenic non-human animal, preferably a mouse. A transgenic non-human animal expressing a polypeptide of the invention is included within the scope of the invention.

It is also possible for the proteins of the invention to be transiently expressed in a cell line or on a membrane, such as for example in a baculovirus expression system. Such systems, which are adapted to express the proteins according to the invention, are also included within the scope of the present invention.

According to another aspect, the present invention also relates to antibodies (either polyclonal or preferably monoclonal antibodies, chimeric, single chain, Fab fragments) which have been raised by standard techniques and are specific for a polypeptide of the invention. Such antibodies could for example, be useful in purification, isolation or screening methods involving immunoprecipitation techniques and may be used as tools to further elucidate the function of TLR9 or a variant thereof, or indeed as therapeutic agents in their own right. Antibodies may also be raised against specific epitopes of the proteins according to the invention. Such antibodies may be used to block ligand binding to the receptor. Alternatively an antibody may be provided which acts as an agonist, to cross link receptors of the invention to mediate receptor activity. An antibody, or other compound, “specifically binds” to a protein when it binds with high affinity to the protein for which it is specific but does not bind or binds with only low affinity to other proteins. A variety of protocols for competitive binding or immunoradiometric assays to determine the specific binding capability of an antibody are well known in the art (see for example Maddox et al 1993). Such immunoassays typically involve the formation of complexes between the “specific protein” and its antibody and the measurement of complex formation.

An important aspect of the present invention is the use of polypeptides according to the invention in screening methods to identify compounds that may act as modulators of Toll-like receptor activity. Any suitable form may be used for the assay to identify a modulator of TLR9 activity. In general terms, such screening methods may involve contacting a polypeptide of the invention with a test compound and then measuring receptor activity.

Screening methods may alternatively involve contacting a polypeptide of the invention with a test compound and then monitoring for the effect on immunomodulatory activity.

The binding of the substance to a polypeptide in the invention can be determined directly. For example, a radiolabelled test substance can be incubated with a polypeptide of the invention and so that binding of the test substance to the polypeptide can be monitored. Typically, the radiolabelled test substance can be incubated with cell membranes containing the polypeptide until equilibrium is reached. The membranes can then be separated from a non-bound test substance and dissolved in scintillation fluid to allow the radioactive content to be determined by scintillation counting. Non-specific binding of the test substance may also be determined by repeating the experiments in the presence of a saturating concentration of a non-radioactive ligand. Preferably, a binding curve is constructed by repeating the experiment with various concentrations of the test substance.

Cell based assays may also be carried out, for example using a cell expressing the TLR9 receptor, and contacting the cell with another cell to look for ligand binding or activation of TLR9-mediated pathways such as NFκB activation. Alternatively cells expressing TLR9 constitutively may be provided for use in assays for TLR9 function. Such constitutively expressed TLR9 may demonstrate TLR9 activity in the absence of ligand binding. Additional test substances may be introduced in any assay to look for inhibitors of ligand binding or inhibitors of TLR9-mediated activity. Assays are preferably carried out using cells expressing TLR9, and incubating such cells with the test substance optionally in the presence of TLR9 ligand. Alternatively an antibody may be used to complex TLR9 and thus mediate TLR9-activity. Test substances may then be added to assess the effect on such activity.

In preferred aspects, a host cell is provided expressing the receptor, or a receptor complex of TLR9 (or TLR9-A) comprising a homodimer, a heterodimer of TLR9 (or TLR9-A) with another Toll-like receptor, or a complex of TLR9 (or TLR9-A) with protein cofactors, and containing an NFκB responsive reporter construct. The host cell is treated with a substance under test for a defined time. The expression of the reporter gene, such as secreted placental (SP) alkaline phosphatase or luciferase is assayed. The assay enables determination of whether the addition of compounds inhibits the induction of the response in target cells. Alternatively the assay may be carried out to identify cytokine production or it may be carried out in the presence of T-cells to identify inducement of co-stimulatory receptors required for activation of T-cells.

Assays may also be carried out to identify modulators of receptor shedding. A polypeptide of the invention can be cleaved from the cell surface. Shedding the receptor would act to down regulate receptor signalling. Thus, cell based assays may be used to screen for compounds which promote or inhibit receptor-shedding. Assays may also be carried out to identify substances which modify TLR9 receptor expression for example substances which down regulate expression. Such assays may be carried out for example by using antibodies for TLR9 to monitor levels of TLR9 expression.

Additional control experiments may be carried out. Assays may also be carried out using known ligands of other Toll-like receptors to identify ligands which are specific for polypeptides of the invention. Preferably, the assays of the invention are carried out under conditions which would result in immunomodulatory NFκB mediated activity in the absence of the test substance, to identify inhibitors of Toll-like receptor mediated activity, or agents which inhibit ligand-induced Toll-like receptor activity.

Suitable test substances which can be tested in the above assays include combinatorial libraries, defined chemical entities, peptides and peptide mimetics, oligonucleotides and natural product libraries, such as display (e.g. phage display libraries) and antibody products. In a preferred embodiment, the test substance is a variant peptide of the invention. In particularly preferred embodiments, suitable test substances which may be candidate ligands for binding to and modulation of TLR9 or TLR9-A include synthetic or naturally occurring oligonucleotides or polynucleotides which bind directly to the Toll-like receptor or which modulate the Toll-like receptor of the present invention via one or more intermediate signal transducers. Such oligo- or polynucleotides may be “CG-rich” sequences or sequences including one or more unmethylated CpG nucleotide pairs.

The assay may be carried out using full length TLR9 to identify a variant peptide which interferes with TLR9 mediated activity, for example by inhibiting ligand binding.

Test substances may be used in an initial screen of, for example, 10 substances per reaction, and the substances of these batches which show inhibition or activation tested individually. Test substances may be used at a concentration of from 1 nM to 1000 μM, preferably from 1 μM to 100 μM, more preferably from 1 μM to 10 μM.

A protein-binding assay may be developed using a polypeptide of the invention, preferably one encoding the extracellular ligand-binding domain, to identify novel protein ligands of TLR9. Particular examples may be screening of a human cDNA expression library for protein ligands of TLR9 by yeast 2-hybrid protein interaction assay (e.g. as described in International Patent Application No. WO99/49294).

Another aspect of the present invention is the use of polynucleotides encoding the TLR9 polypeptides of the invention to identify mutations in TLR9 genes which may be implicated in human disorders or to identify cells in which TLR9 is expressed. Identification of such mutations may be used to assist in diagnosis of immune system, lung, kidney, heart or other disorders or susceptibility to such disorders and in assessing the physiology of such disorders. In particular the polynucleotides of the invention may assist in diagnosis of asthma and rheumatoid arthritis. For example, a SNP (single nucleotide polymorphism) has been identified in the genomic DNA encoding TLR9 (G/A nucleotide: The SNP Consortium database accession number TSC0164834). This single base pair change lies in the DNA region encoding the 23 N-terminal residues of TLR9, and this region is spliced out of the mRNA encoding TLR9-A. The nucleotide at this SNP position may affect the efficiency of mRNA splicing in the two different variants—a G at this position may possibly disrupt the splicing machinery and an A might lead to more efficient splicing. Additionally, the presence of a G as compared to an A in an unspliced mRNA would introduce a stop codon and result in different N terminal protein sequences upon translation of that mRNA, thus the two polymorphic variants of the tlr9 gene may encode receptors which have differing expression levels and/or differing functional activity levels. The present invention provides a diagnostic tool for determining the polymorphic variant in an individual by detecting the DNA sequence at the SNP site. Such a tool may incorporate a nucleotide probe specific for one or other of the polymorphic variants, for example an oligonucleotide of from 5 to 50, preferably 5-20 nucleotides, complementary to a fragment of the nucleotide sequence of SEQ ID NO. 1 which extends over the SNP site or a fragment complementary to that sequence with the exception of the single nucleotide change (G to A) at the SNP site. The present invention also provides a method of detecting a polymorphic variant in the tlr9 gene by determining the sequence of nucleotides at and around the SNP site identified by the SNP consortium database accession number TSC0164834, in particular by determining whether the nucleotide at that SNP site is a G or an A.

Another aspect of the present invention is the use of the compounds that have been identified by screening techniques referred to above in the treatment or prophylaxis of disorders which are responsive to regulation of TLR9 receptor activity. In addition, variant peptides of the invention which inhibit TLR9-mediated activity, for example which inhibit ligand binding or prevent hTLR9 immunomodulatory activity may be used in the treatment or prophylaxis of such disorders. Antibodies which recognise TLR9 may similarly be used in therapy.

In particular, such compounds may be used in the treatment of inflammatory, cardiovascular, systemic infection or autoimmune disease. The compounds may be used to treat bacterial, viral or fungal infections, asthma, rhinitis, chronic obstructive pulmonary disease (COPD), emphysema, an inflammatory bowel disease such as ulcerative colitis and Crohn's disease, rheumatoid arthritis, osteoarthritis, psoriasis, Alzheimers disease, atherosclerosis, septic shock syndrome associated with systemic infection involving gram positive and gram negative bacteria, diabetes, Multiple Sclerosis.

In an alternative aspect, the invention provides agents which activate TLR9 mediated immunomodulation for use as an immunoadjuvant, or TLR9, and variants thereof, or polynucleotides or DNA encoding a polypeptide of the invention which may be administered for use as immunoadjuvants to enhance or alter the immune response in an individual to an antigen.

The compounds identified according to the screening methods outlined above may be formulated with standard pharmaceutically acceptable carriers and/or excipients as is routine in the pharmaceutical art, and as fully described in Remmington's Pharmaceutical Sciences, Mack Publishing Company, Eastern Pennsylvania 17^{th} Ed. 1985, the disclosure of which is included herein of its entirety by way of reference.

The compounds may be administered by enteral or parenteral routes such as via oral, buccal, anal, pulmonary, intravenous, intra-arterial, intramuscular, intraperitoneal, topical or other appropriate administration routes. The dose of a compound to be administered may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the compound, the age, weight and conditions of the subject to be treated, the type and severity of the disease and the frequency and route of administration. Preferably, daily dosage levels are from 5 mg to 2 g.

Nucleic acid encoding an inhibitor of TLR9 activity may be administered to the mammal. In an alternative aspect of the invention, nucleic acid encoding TLR9 or a variant thereof may be administered to the animal. Such a variant shows immunomodulatory activity of TLR9 such as inducing cytokine production and expression of cell surface co-stimulatory receptors required for activation of T-cells. Nucleic acid, such as RNA or DNA, and preferably, DNA, is provided in the form of a vector, such as the polynucleotides described above, which may be expressed in the cells of the mammal.

Nucleic acid encoding the peptide may be administered to the animal by any available technique. For example, the nucleic acid may be introduced by injection, preferably intradermally, subcutaneously or intramuscularly. Alternatively, the nucleic acid may be delivered directly across the skin using a nucleic acid delivery device such as particle-mediated gene delivery. The nucleic acid may be administered topically to the skin, or to the mucosal surfaces for example by intranasal, oral, intravaginal, intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 μg nucleic acid for particle mediated gene delivery and 10 μg to 1 mg for other routes.

### EXAMPLES

### Example 1

### Screening for Compounds Which Exhibit Protein Modulating activity

Mammalian cells, such as Hek293, CHO and HeLa cells over-expressing TLR9 protein are generated for use in the assay. 96 and 384 well plate, high throughput screens (HTS) are employed. TLR9 induced cell activation can be monitored through activation of a signal transduction event (typically resulting in activation of NFκB or AP-1) or transcriptional activation of a reporter gene (typically regulated via NFκB or AP-1 responsive elements). TLR9 induced activation of a reporter gene such alkaline phosphatase or luciferase can be easily assessed using a suitable colourimetric or fluorimetric assay to measuring production. Such assays may be used to identify receptor antagonists capable of blocking ligand induced TLR9 activation, inhibitors capable of blocking TLR9 intracellular signal transduction or receptor agonists capable of activating TLR9. Secondary screening involves evaluation of TLR9 modulators in disease related cells. Tertiary screens involve the study of modulators in rat and mouse models of disease relevant to the target.

1. An isolated Toll-like-receptor polypeptide consisting essentially of
(i) the amino acid sequence SEQ ID NO: 2;
(ii) a variant thereof which has immunomodulatory activity; or
(iii) a fragment of (i) or (ii) which has immunomodulatory activity.

2. A polypeptide according to claim 1 wherein the variant (ii) has at least 70% identity to the amino acid sequence of SEQ ID NO: 2.

3. A polypeptide according to claim 2 which has at least 70% identity to the amino acid sequence of SEQ ID NO: 2 over a region of at least 70% of the full-length sequence provided by SEQ ID NO. 1 and exhibits toll-like receptor functionality.

4. A polypeptide according to claim 1 or claim 2 wherein the variant (ii) has at least 95% identity to the amino acid sequence of SEQ ID NO: 2.

5. A polypeptide according to claim 2 which has at least 95% identity to the amino acid sequence of SEQ ID NO: 2 over a region of at least 60 contiguous amino acids and exhibits toll-like receptor functionality.

6. A polypeptide according to claim 1 wherein the fragment (iii) is a peptide of up to 150 amino acids in length and exhibits toll-like receptor functionality.

7. A polynucleotide encoding a polypeptide according to any one of claims 1-3.

8. A polynucleotide encoding a Toll-like receptor polypeptide which has immunomodulatory activity, which polynucleotide consists essentially of:
(a) the nucleic acid sequence of SEQ ID NO: 1;
(b) a sequence complementary thereto;
(c) a sequence which hybridises under stringent conditions to a sequence as defined in (a) or (b);
(d) a sequence that is degenerate as a result of the genetic code to a sequence as defined in (a), (b) or (c); or
(e) a sequence having at least 60% identity to a sequence as defined in (a), (b), (c) or (d).

9. A polynucleotide according to claim 7 or claim 8 which is mRNA.

10. A polynucleotide according to claim 7 or claim 8 which is DNA.

11. A polynucleotide according to claim 7 or claim 8 which is cDNA.

12. An isolated toll-like receptor polypeptide which is obtainable by expression in vivo or in vivo of a polynucleotide according to claim 7 or claim 8.

13. A polypeptide according to claim 12 which has the structural features conserved amongst toll-like receptors.

14. An expression vector comprising a polynucleotide sequence according to any one of claims 7 to 11, which is capable of expressing a polypeptide according to any one of claims 1 to 3 or claim 12.

15. An expression vector according to claim 14 which is a plasmid, phage or viral vector.

16. A host cell comprising an expression vector according to claim 14 or claim 15.

17. A polyclonal or monoclonal antibody, or a chimera or fragment thereof, which is specific for a polypeptide according to any one of claims 1 to 3.

18. A method for identification of a compound that modulates Toll-like receptor activity, which method comprises contacting a polypeptide according to any one of claims 1 to 3 or claim 12 with a test substance and monitoring for immunomodulatory activity.

19. A compound which modulates Toll-like receptor activity and which is identifiable by a method according to claim 18.

20. A compound according to claim 19 which is a peptide or polypeptide.

21. A compound according to claim 19 which is an oligonucleotide or polynucleotide.

22. A method of treating a subject having an inflammatory or cardiovascular disorder, systemic infection or autoimmune disease that is responsive to Toll-like receptor modulation, which method comprises administering to said subject an effective amount of a compound according to any one of claims 19 to 21 or an antibody according to claim 17.

23. A method according to claim 22 wherein the disorder is a viral, fungal or bacterial infection, asthma, rhinitis, chronic obstructive pulmonary disease (COPD), emphysema, an inflammatory bowel disease such as ulcerative colitis or Crohn's disease, rheumatoid arthritis, osteoarthritis, psoriasis, Alzheimers disease, atherosclerosis, Multiple Sclerosis, diabetes or septic shock syndrome associated with systemic infection involving gram positive or gram negative bacteria.

24. A polypeptide comprising a fragment or variant of SEQ ID NO: 2, which is capable of inhibiting the activity of TLR9 having the amino acid sequence of SEQ ID NO: 2 or a functional variant thereof, for use in the treatment of an immune or inflammatory disorder.

25. A polypeptide according to any one of claims 1 to 3 or claim 12, a polynucleotide according to claim 7 or claim 8 or a compound according to claim 19 for use as an adjuvant.

26. The use of a compound according to claim 19 in the manufacture of a medicament for the treatment of an immune or inflammatory disorder.

27. The use of a polypeptide comprising a fragment or variant of SEQ ID NO: 2, which is capable of inhibiting the activity of TLR9 having the amino acid sequence of SEQ ID NO: 2 or a functional variant thereof, in the manufacture of a medicament for the treatment of an immune or inflammatory disorder.

28. The use of a polypeptide according to any one of claims 1 to 3 or claim 12, a polynucleotide according to claim 7 or claim 8 or a compound according to claim 19 in the manufacture of an adjuvant or vaccine formulation.
